(19) United States
(12) Patent Application Publication
(10) Pub. No.: US 2017/0312113 A1
(43) Pub. Date: Nov. 2, 2017

(54) ANCHOR FOR USE WITH PATIENT RESTRAINTS AND ASSOCIATED METHODS

(71) Applicant: J.T. Posey Company, Arcadia, CA (US)

(72) Inventors: Eduardo Acuna, Azusa, CA (US); Charles R. Kline, Ontario, CA (US)

(21) Appl. No.: 15/140,308

(22) Filed: Apr. 27, 2016

(51) Int. Cl. A61F 5/37; A44B 11/20

(52) U.S. Cl. CPC A61F 5/3769; A61F 5/3776; A44B 11/20; A61F 5/3761

(57) ABSTRACT

A restraint anchor in accordance with at least one of the present inventions includes an attachment member that is adapted to be secured to a patient restraint and a base member, associated with the attachment member, that is adapted to be secured to a portion of a patient support apparatus.

### BACKGROUND

### 1. Field

The present inventions relate generally to patient restraints.

### 2. Related Art

Patient restraints are commonly used to secure one or more portions of a patient's body (e.g., the patient's limbs) to a hospital bed, gurney, stretcher or the like (collectively referred to herein as a “patient support apparatus”) so as to prevent patients from injuring themselves or others. Patient support apparatus manufactured in years past frequently included anchor points for the restraints that were readily accessible when a patient was on the patient support apparatus. When restraint was necessary, one portion of a restraint could be secured to the patient and another portion could be secured to the anchor point. More recently manufactured patent support apparatus tend to lack such anchor points.

The present inventors have determined that the lack of anchor points on patent support apparatus is problematic for a number of reasons. For example, patients often must be restrained quickly due to medical necessity. When a patient on a hospital bed is in need of restraint, caregivers must remove the patient from the hospital bed, remove the mattress from the hospital bed frame, secure the restraints to the frame or undercarriage, replace the mattress, and place the patient back on the bed prior to securing the restraints to the patient. Alternatively, the caregivers may be forced to lift various portions of the mattress and secure the restraints to the frame or undercarriage while the patient is on the bed, or to simply secure the restraints to a side rail or other less desirable portion of associated patent support structure. As such, the lack of a readily accessible access point subjects the caregivers and patients to delay and the possibility of injury.

### SUMMARY

A restraint anchor in accordance with at least one of the present inventions includes an attachment member that is adapted to be secured to a patient restraint and a base member, associated with the attachment member, that is adapted to be secured to a portion of a patient support apparatus.

A restraint anchor in accordance with at least one of the present inventions includes means for anchoring a patient restraint and means for securing the means for anchoring to a portion of a patient support apparatus.

A method in accordance with at least one of the present inventions includes the step of securing a restraint anchor, which includes an attachment member that is adapted to be secured to a patient restraint, to the patient support apparatus.

### BRIEF DESCRIPTION OF THE DRAWINGS

Detailed description of preferred embodiments of the inventions will be made with reference to the accompanying drawings.

FIG. 1 is a perspective view of a restraint anchor in accordance with one embodiment of a present invention.

FIG. 2 is a perspective view of a portion of the restraint anchor illustrated in FIG. 1.

FIG. 3 is a perspective view of a portion of the restraint anchor illustrated in FIG. 1.

FIG. 4 is a bottom view of a portion of the restraint anchor illustrated in FIG. 1.

FIG. 5 is a perspective view of the restraint anchor illustrated in FIG. 1 with the base member in a looped state.

FIG. 6 is a perspective view of a conventional patient support apparatus.

FIG. 7 is a perspective view of a portion of the conventional patient support apparatus illustrated in FIG. 6 with the mattress removed.

FIG. 8 is a perspective view of a portion of the conventional patient support apparatus illustrated in FIG. 6, with the mattress removed, a portion of the support deck pivoted away from the frame, and the exemplary restraint anchor secured thereto.

FIG. 9 is a perspective view showing a patient restraint anchored to the exemplary restraint anchor.

### DETAILED DESCRIPTION OF THE EXEMPLARY EMBODIMENTS

The following is a detailed description of the best presently known modes of carrying out the inventions. This description is not to be taken in a limiting sense, but is made merely for the purpose of illustrating the general principles of the inventions.

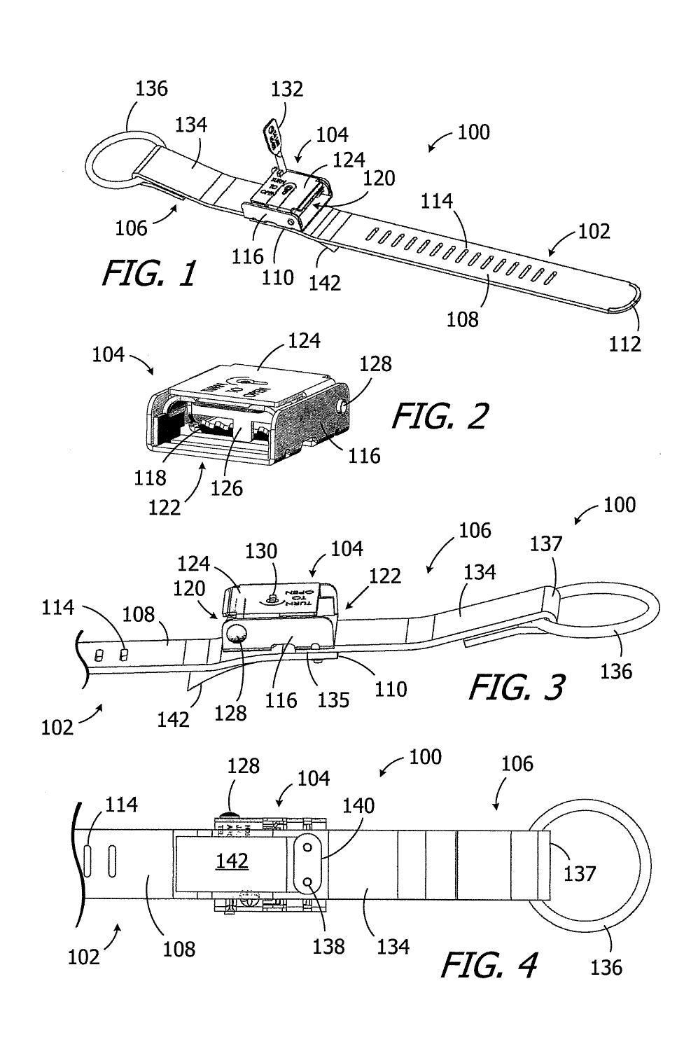

A restraint anchor in accordance with one embodiment of the present inventions is generally represented by reference numeral 100 in FIGS. 1-5. The exemplary restraint anchor 100 includes a base member 102 that may be looped around a portion of the bed frame or undercarriage in the manner described below, a buckle 104 or other fastening apparatus that maintains the base member in the looped state, and an attachment member 106 to which a patient restraint may be secured.

The exemplary base member 102 includes a flexible strap 108, with a fixed end 110 that is secured to the buckle 104 and a free end 112, and a plurality of apertures 114 that extend through the flexible strap.

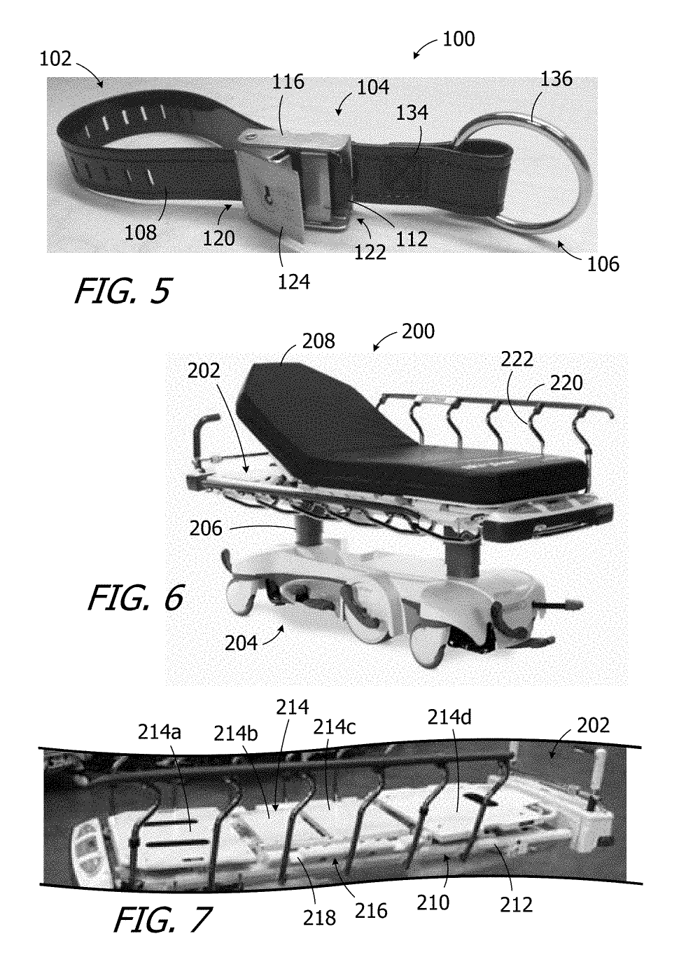

The exemplary buckle 104 includes a housing 116 with an internal row of teeth 118, an inlet 120 and an outlet 122. A pivotable cover 124, which is movable between a closed position (FIGS. 1-3) and an open position (FIG. 5), carries a post 126 and is connected to the housing 116 by an axle 128. The exemplary buckle 104 includes a lock (not shown) that maintains the buckle in the closed position. A keyway 130 is provided for a key 132 that may be used to lock and unlock the buckle 104. The lock may be omitted from the buckle in other implementations. The exemplary buckle 104 may also be replaced by a quick release buckle, or by hook and loop fastener material on portions of the flexible strap 108.

The exemplary attachment member 106 includes a flexible strap 134 with a fixed end 135 that is secured to the buckle 104 and a free end 137. An anchor ring 136, which provides a location at which the patient restraint may be connected to the restraint anchor 100, is secured to the free end 137. The anchor ring 136, which may be formed from metal, plastic or other suitable materials, may have a circular shape (as shown), a D-shaped or any other suitable shape. In other implementations, the anchor ring may be in the form of a sewn loop formed at the end of the flexible strap 134.

Referring more specifically to FIGS. 3 and 4, the flexible straps 108 and 134 in the illustrated embodiment may be secured to one another and to the buckle 104 with rivets 138 and a plate 140. In other embodiments, the flexible straps 108 and 134 may be combined into a single flexible member with portions on opposite side of the buckle 104 that respectively define the flexible straps of the base member and attachment member. The rivets 138 and a plate 140 may also be used to secure a tag 142 to the buckle 104.

The free end 112 of the flexible strap 108 may be inserted into the buckle housing inlet 120, through the buckle housing 116 past the post 126 (FIG. 2) and out of the outlet 122, in the manner illustrated in FIG. 5, when the buckle 104 is in the open state with the pivotable cover 124 in the open position. The buckle 104 may be closed by pivoting the cover 124 to the closed position (FIG. 2), which drives the post 126 into one of the apertures 114 and a portion of the flexible strap 108 against the teeth 118 to secure a second portion of the looped flexible strap 108 to the buckle. The buckle 104 may then be locked

Although the present restraint anchors are not limited to use with any particular patient support apparatus, one example of a patient support apparatus that may be used in conjunction with the present restraint anchor 100 is the patient support apparatus 200 illustrated in FIGS. 6 and 7. Briefly, the patient support apparatus 200 includes a support litter 202, a wheeled base 204 and an elevation mechanism 206. The wheeled base 204 and elevation mechanism 206 define the undercarriage of the patient support apparatus 200. A mattress 208 is carried on the support litter 202. The support litter 202 includes a frame 210 with support rails 212, a patient support deck 214 including deck members 214a-214d, and resilient side bolsters 216 (with indentations 218) that are located between the support rails 212 and the support deck 214. The patient support apparatus 200 also includes side rails 220 that are carried on pivotable support arms 222. The patient support apparatus 200 does not, however, include an anchor point that is accessible when a patient is on the mattress 208. One example of such a patient support apparatus is illustrated and described in U.S. Pat. No. 7,124,456, which is incorporated herein by reference in its entirety.

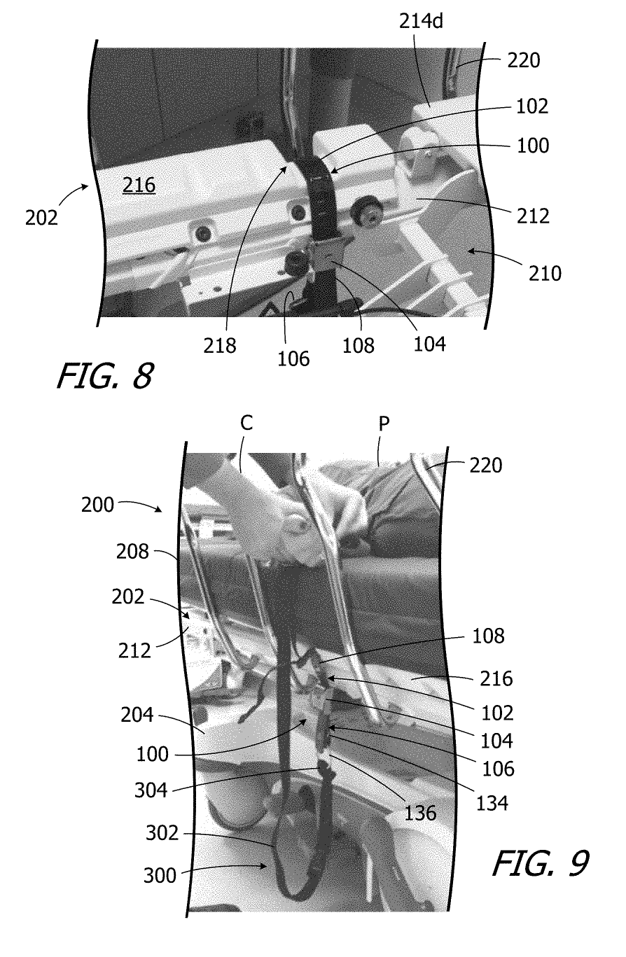

One or more of restraint anchors 100 may be secured to the patient support apparatus 200 to convert the patient support apparatus into a patient support apparatus that has one or more anchor points in, for example, the manner described here with reference to FIG. 8. When the patient support apparatus 200 is not occupied by a patient, the mattress 208 may be removed and the deck member 214c may be pivoted off the side bolsters 216. Other deck members may be moved (or not moved) when the restraint anchor is to be secured to a different portion of the patient support apparatus. The base member 102 may then be secured to the frame 210 by wrapping (or “looping”) the flexible strap 108 around the support rail 212 and/or any other appropriate structure with the appropriate size and strength. In those instances where there are additional structures associated with the support rail, such as the side bolster 216 in the illustrated example, the flexible strap 108 may also be wrapped around those structures. Here, the flexible strap 108 is looped around the support rail 212 and side bolster 216, and is positioned within the indentation 218 to prevent movement along the length of the support rail 212. Next, with the buckle 104 in the open state, the free end 118 of the strap 108 may be inserted through the buckle 104 in the manner discussed above with reference to FIG. 5 and pulled until the base member 102 is tightly looped around the support rail 212 and bolster 116. The buckle 104 may then be closed and (if desired) locked, thereby creating an anchor point that will be accessible when a patient is on the mattress 208.

This process may be repeated to place additional restraint anchors 100 at other locations on the patient support apparatus 200. By way of example, but not limitation, four restraint anchors 100 may be secured to the patient support apparatus 200 at locations generally corresponding to the patient's wrists and ankles.

It should also be noted here that the configuration of the present restraint anchors 100 allows the restraint anchors to be removably secured to the patient support apparatus. As used here, the phrase “removably secured” means that a restraint anchors can be detached from a patient support apparatus without damage to the restraint anchor or the patient support apparatus.

The orientation of the restraint anchor 100 relative to the patient support apparatus to which it is attached will depend on the configuration of the patient support apparatus and the most convenient locations for the attachment member 106. In the illustrated example, the attachment member 106 extends downwardly from the frame 210 into the open space below the fame in the manner illustrated in FIG. 9. So located, the restraint anchor 100 (and the attachment member 106 in particular) will be readily available for connection to a patient restraint without interfering with other aspects of the operation of the patient support apparatus 200.

A patient restraint 300, which includes an elongate adjustable length strap 302 and a cuff (not shown) for the patient's wrist or ankle, may be secured to the anchor point created by a restraint anchor 100 in the manner illustrated in FIG. 9. The anchor point is defined by the anchor ring 136. After the free end 304 of the strap 302 has been connected to the conveniently located anchor ring 136, the cuff may be secured to the patent P's wrist (as shown) or ankle by the caregiver C. The length of the strap 302 may then be reduced to the appropriate length.

With respect to materials and dimensions, suitable materials for the straps 108 and 134 include, but are not limited to, polyester, nylon, polyurethane, and cotton. The length of the strap 108 may range from about 6 inches to 24 inches, while the length of the strap 134 may range from about 2 inches to 8 inches.

Such lengths would be combined in those instances where a single length of material forms both of the straps 108 and 134. The widths of the straps 108 and 134 are the same in the illustrated embodiment, and may range from about 0.75 inches to 6 inches. The widths may be different in other embodiments. The materials and dimensions are not limited to the examples provided here and may be altered as necessary.

Although the present inventions have been described in terms of the preferred embodiments above, numerous modifications and/or additions to the above-described preferred embodiments would be readily apparent to one skilled in the art. It is intended that the scope of the present inventions extends to all such modifications and/or additions.

We claim:

1. A restraint anchor, comprising:
an attachment member that is adapted to be secured to a patient restraint; and
a base member, associated with the attachment member, that is adapted to be secured to a portion of a patient support apparatus.

2. A restraint anchor as claimed in claim 1, wherein the base member includes a flexible strap defining a free end and a fixed end.

3. A restraint anchor as claimed in claim 2, further comprising:
a buckle associated with the fixed end of the base member flexible strap.

4. A restraint anchor as claimed in claim 1, wherein
the attachment member includes a flexible strap, defining a free end and a fixed end, and a ring associated with the free end.

5. A restraint anchor as claimed in claim 4, further comprising:
a buckle associated with the fixed end of the attachment member flexible strap.

6. A restraint anchor as claimed in claim 1, wherein
the base member is not part of the patient restraint.

7. A restraint anchor, comprising:
means for anchoring a patient restraint; and
means for securing the means for anchoring to a portion of a patient support apparatus.

8. A restraint anchor as claimed in claim 7, further comprising:
a buckle associated with the means for securing.

9. A restraint anchor as claimed in claim 7, wherein
the means for anchoring a patient restraint is not part of the patient restraint.

10. A method of converting a patient support apparatus into a patient support apparatus with an anchor point, comprising the step of:
securing a restraint anchor, which includes an attachment member that is adapted to be secured to a patient restraint, to the patient support apparatus.

11. A method as claimed in claim 10, wherein
the restraint anchor includes a flexible strap; and
the step of the securing a restraint anchor comprises looping the flexible strap around a portion of the patient support apparatus.

12. A method as claimed in claim 11, further comprising the step of:
locking the flexible strap to the portion of the patient support apparatus.

13. A method as claimed in claim 10, wherein
the patient support apparatus includes a frame and a mattress; and
the step of the securing a restraint anchor comprises securing the restraint anchor to the frame.

14. A method as claimed in claim 13, the further comprising the step of:
removing the mattress prior to securing the mattress to the frame.

15. A method as claimed in claim 10, wherein
the attachment member is not part of the patient support apparatus.
